SEQUENCE LISTING (20 sequences)

SEQ ID NO: 1 - DNA, 20 bp, Artificial Sequence
  IPN-gamma real-time PCR forward primer
actggcaaaaggatggtgac

SEQ ID NO: 2 - DNA, 20 bp, Artificial Sequence
  IFN-gama real-time PCR reverse primer
tgagctcattgaatgcttgg

SEQ ID NO: 3 - DNA, 20 bp, Artificial Sequence
  IL-4 real-time PCR forward primer
tcaacccccagctagttgtc

SEQ ID NO: 4 - DNA, 20 bp, Artificial Sequence
  IL-4 real-time PCR reverse primer
tgttcttcgttgctgtgagg

SEQ ID NO: 5 - DNA, 20 bp, Artificial Sequence
  T-bet T-H1-specific transcription factor real-time PCR forward primer
caacaacccctttgccaaag

SEQ ID NO: 6 - DNA, 20 bp, Artificial Sequence
  T-bet T-H1-specific transcription factor real-time PCR reverse primer
tcccccaagcagttgacagt

SEQ ID NO: 7 - DNA, 20 bp, Artificial Sequence
  heme oxygenase 1 (HO-1) real-time PCR reverse primer
cacgcatatacccgctacct

SEQ ID NO: 8 - DNA, 20 bp, Artificial Sequence
  heme oxygenase 1 (HO-1) real-time PCR reverse primer
ccagagtgttcattcgagca

SEQ ID NO: 9 - DNA, 20 bp, Artificial Sequence
  nuclear factor-erythroid-2-related factor 2 (Nrf2) real-time PCR forward primer
ctcgctggaaaaagaagtgg

SEQ ID NO: 10 - DNA, 20 bp, Artificial Sequence
  nuclear factor-erythroid-2-related factor 2 (Nrf2) real-time PCR reverse primer
ccgtccaggagttcagagag

SEQ ID NO: 11 - DNA, 20 bp, Artificial Sequence
  reduced nicotinamide adenine dinucleotide phosphate-quinone oxidoreductase (NQ01) real-time PCR forward primer
ttctctggccgattcagagt

SEQ ID NO: 12 - DNA, 20 bp, Artificial Sequence
  reduced nicotinamide adenine dinucleotide phosphate-quinone oxidoreductase (NQ01) real-time PCR reverse primer
cctgttgcccacaaggtagt

SEQ ID NO: 13 - DNA, 20 bp, Artificial Sequence
  glutathione S-transferase (GST) real-time PCR forward primer
cgccaccaaatatgacctct

SEQ ID NO: 14 - DNA, 20 bp, Artificial Sequence
  glutathione S-transferase (GST) real-time PCR reverse primer
cctgttgcccacaaggtagt

SEQ ID NO: 15 - DNA, 20 bp, Artificial Sequence
  gamma-glutamylcysteine ligase (gamma-GCL, gamma-GCLS) real-time PCR forward primer
tggagcagctgtatcagtgg

SEQ ID NO: 16 - DNA, 21 bp, Artificial Sequence
  gamma-glutamylcysteine ligase (gamma-GCL, gamma-GCLS) real-time PCR reverse primer
atgagcagttctttcgggtca

SEQ ID NO: 17 - DNA, 20 bp, Artificial Sequence
  glutathione peroxidase (GPx) real-time PCR forward primer
gtccaccgtgtatgccttct

SEQ ID NO: 18 - DNA, 20 bp, Artificial Sequence
  glutathione peroxidase (GPx) real-time PCR reverse primer
tctgcagatcgttcatctcg

SEQ ID NO: 19 - DNA, 19 bp, Artificial Sequence
  beta-actin reference gene real-time PCR forward primer
agccatgtacgtagccatc

SEQ ID NO: 20 - DNA, 19 bp, Artificial Sequence
  beta-actin reference gene real-time PCR reverse primer
ctctcagctgtggtggtga

(19) United States
(12) Patent Application Publication
(10) Pub. No.: US 2013/0344106 A1
(43) Pub. Date: Dec. 26, 2013

(54) Methods and Compositions for Improving Immune Response by a Nutraceutical Antioxidant

(71) Applicants: ANDRE E. NEL, SHERMAN OAKS, CA (US); HYON-JEEN KIM, LOS ANGELES, CA (US)

(72) Inventors: ANDRE E. NEL, SHERMAN OAKS, CA (US); HYON-JEEN KIM, LOS ANGELES, CA (US)

(21) Appl. No.: 13/907,799

(22) Filed: May 31, 2013

Related U.S. Application Data
(62) Division of application No. 12/921,113, filed on Apr. 22, 2011, now abandoned.
(60) Provisional application No. 61/033,688, filed on Mar. 4, 2008.

(51) Int. Cl. A61K 39/39

(52) U.S. Cl. CPC A61K 39/39
USPC 424/209.1; 424/278.1; 424/224.1; 424/226.1; 424/227.1; 424/228.1; 424/204.1; 424/212.1; 424/211.1; 424/219.1; 424/245.1; 424/240.1; 424/239.1; 424/230.1; 424/215.1; 424/250.1

(57) ABSTRACT

The present invention provides a new means of restoring the immune system in aging and immunocompromised individuals using an antioxidant nutraceutical. The nutraceutical stimulates the aging immune system through the Nrf2 master gene regulatory pathway. The invention is based in part on the discovery that the Nrf2 has antioxidant and immune restorative activity. The nutraceutical improves function of both the innate and adaptive immune systems.

### CROSS-REFERENCES TO RELATED APPLICATIONS

This application claims priority to U.S. Provisional Appl. No. 61/033,688, filed Mar. 4, 2008.

### STATEMENT AS TO RIGHTS TO INVENTIONS MADE UNDER FEDERALLY SPONSORED RESEARCH OR DEVELOPMENT

This invention was made under government support under Grant Award Nos. AG014992 and A1090453 awarded by the NIH. The government has rights in this invention.

### BACKGROUND OF THE INVENTION

Age related immune function decline is becoming an increasingly serious problem. Immune senescence is an important topic from the perspective of aging demographics and the associated increase in infectious disease episodes. The functional decline results in high susceptibility to viral and microbial infection and higher incidence and progression of cancer.

The decrease in cellular immunity with aging is of considerable public health importance. Vaccines have a huge failure rate in the elderly. No one has studied the effect of altering the redox equilibrium in the immune system, and specifically in antigen presenting cells (APC), on immune function in the elderly.

A decrease in T_{H}1 immunity with aging is of particular importance in defense against viral and mycobacterial pathogens, as well as for immune surveillance against cancer. Although a host of specific molecular and cellular events have been described in senescent immune cells (Fulop et al., Arthritis Res Ther, 5:290-302 (2005)), it is not clear whether aging is responsible for a common mechanism of immune decrease. Harman's original free radical theory suggested that aging could be attributed to the deleterious effects of reactive oxygen species (ROS) (Harman, J Gerontol, 11:298-300 (1956)).

ROS are byproducts of normal processes, such as the metabolic conversion of food into energy and can also enter the body through small particles present in polluted air. They can cause oxidative tissue damage leading to disease—such as triggering the inflammation process that causes clogging of arteries. Oxidative tissue damage of body tissues and organs probably constitutes one of the major reasons why we age.

Although it is known that ROS can damage structural cellular components and can induce a state of oxidative stress by means of glutathione (GSH) depletion, it is not intuitive how disrupting redox equilibrium could induce immune effects. It is possible that lower levels of oxidative stress induce a protective and adaptive antioxidant defense that allows oxidant injury to become manifest only when this defense is overcome by high levels of ROS production (Xiao et al., J Biol Chem, 278:50781-90 (2003)).

We are beginning to understand that oxidative stress is not just confined to oxidant injury, and to consider antioxidant defense mechanisms. In fact, the coordinated antioxidant defense that is initiated by the Nrf2 pathway is the most sensitive oxidative stress response (Itoh et al., Biochem Biophys Res Common, 236:313-22 (1997)).

Nrf2 (Nuclear Factor-Erythroid-2-Related Factor 2) is a member of the CNC family of bZIP transcription factors. Nrf2 regulates the transcriptional activation of more than 200 antioxidant and protective genes that constitute the phase II response. Examples of phase II enzymes (p2Es) include the rate-limiting enzyme in the GSH synthesis pathway, γ-glutamylcysteine ligase (γ-GCL), as well as glutathione peroxidase (GPx), heme oxygenase 1, superoxide dismutase, glutathione S-transferase, and reduced nicotinamide adenine dinucleotide phosphate-quinone oxidoreductase (NQO1).

Antioxidants unrelated to the Nrf2 pathway have previously been applied to immune dysfunction with marginal success (see, e.g., Meydani et al. (1990) Am J Clin Nutr 52:557-63). These studies generally do not focus on APCs. The role of Nrf2 in restoring intracellular redox equilibrium in aging populations, and for adaptive immunity in particular, has not previously been investigated.

Recent studies suggest that the redox equilibrium of dendritic cells (DCs) is a key factor in maintaining protective cellular immunity and that a disturbance of this homeostatic mechanism could contribute to immune senescence. The present invention provides a new means of restoring the immune system in aging individuals using an antioxidant nutraceutical. The nutraceutical stimulates the aging immune system through the Nrf2 master gene regulatory pathway. The present invention is based in part on the discovery that the Nrf2 has antioxidant and immune restorative activity. The nutraceutical improves function of both the innate and adaptive immune systems.

### BRIEF SUMMARY OF THE INVENTION

The invention provides methods and compositions for improving the immune function and efficacy of vaccination for older and/or immunocompromised individuals. Such methods and compositions are aimed at restoring redox equilibrium in immune cells, e.g., using compounds such as Nrf2. N-acetyl cysteine (NAC), phase II enzymes (p2Es), and Nrf2 pathway agonists (NPAs).

In some embodiments, the invention provides methods of improving immune function in an individual in need thereof, comprising: administering to the individual a nutraceutical composition comprising an effective amount of a Nrf2 pathway agonist (NPA), thereby improving immune function in the individual.

In some embodiments, the NPA is selected from the group consisting of sulforaphane, glucoraphanin, α-linoic acid, or an NPA-containing food or food extract. In some embodiments, the NPA is extracted from a plant, e.g., broccoli, or another cruciferous vegetable. In some embodiments, the nutraceutical is administered orally, e.g., in a tablet, capsule, powder, or liquid.

In some embodiments, the improvement in immune function is an increase in T_{H}1 function, e.g., increased expression of T_{H}1 related genes and cytokines. In some embodiments, the improvement in immune function is an increase in p2E expression in immune cells, e.g., T_{H}1 cells and dendritic cells (DCs). In some embodiments, the improvement in immune function is improved recruitment of DCs and other antigen presenting cells (APCs) to sites of injury, inflammation, or other immune assault. In some embodiments, the improvement in immune function is improved APC activity in DCs. In some embodiments, the improvement in immune function is an improved antigen response, e.g., after exposure to a pathogen or vaccine. In some embodiments, the improvement is in innate immunity.

In some embodiments, the individual in need of improved immune function is immunocompromised. In some embodiments, the individual in need thereof is at risk of reduced immune function. In some embodiments, the individual is at an increased risk of infection. In some embodiments, the individual is at least 45, 50, 55, 60, 65, 70, or 75 years old.

In some embodiments, the method further comprises monitoring immune function in the individual. In some embodiments, the method further comprises monitoring redox equilibrium in an immune cell, e.g., a DC, of the individual. In some embodiments, redox equilibrium is monitored by detecting thiol levels. In some embodiments, the redox equilibrium is monitored by detecting the level of reactive oxygen species (ROS).

The invention also provides methods and compositions for vaccination of individuals. Accordingly, the invention provides methods of improving the efficacy of a vaccine in an individual in need thereof, comprising: administering to the individual a nutraceutical composition comprising an effective amount of a Nrf2 pathway agonist (NPA), thereby improving the efficacy of the vaccine in the individual. In some embodiments, the nutraceutical is administered at the same time, in combination with, the vaccine, e.g., as an adjuvant. In some embodiments, the nutraceutical and vaccine are administered by inhalation, transdermal means, or injection.

In some embodiments, the nutraceutical is administered separately. In some embodiments, the nutraceutical is administered on a separate schedule from the vaccine, e.g., serially. In some embodiments, the nutraceutical is administered orally while the vaccine is administered by inhalation, transdermal means, or injection.

In some embodiments, the NPA is selected from the group consisting of sulforaphane, glucoraphanin, α-linoic acid, or an NPA-containing food or food extract. In some embodiments, the NPA is extracted from a plant, e.g., broccoli, or another cruciferous vegetable. In some embodiments, the vaccine is an influenza vaccine. In some embodiments, the vaccine is selected from the group consisting of: rabies, hepatitis A, hepatitis B, hepatitis C, human papilloma virus, polio, mumps, measles, rubella, diphtheria, pertussis, tetanus, HiB, chickenpox, rotavirus, influenza, meningococcal disease, and pneumonia.

In some embodiments, the invention provides pharmaceutical compositions comprising a combination of (i) a nutraceutical composition comprising an effective amount of a Nrf2 pathway agonist (NPA) and (ii) a vaccine. In some embodiments, the pharmaceutical composition further comprises an adjuvant. In some embodiments, the pharmaceutical composition comprises more than one vaccine. In some embodiments, the pharmaceutical composition comprises more than one NPA, or an NPA from more than one source. In some embodiments, the composition further comprises an additional compound to restore redox equilibrium, such as NAC.

In some embodiments, the invention provides uses for medicaments and methods of manufacture to improve immune function or improve the efficiency of a vaccine in an individual in need thereof. Such medicaments comprise nutraceutical composition comprising an effective amount of a Nrf2 pathway agonist (NPA) or said nutraceutical in combination with a vaccine.

The invention further provides methods of identifying a Nrf2 pathway agonist (NPA), said method comprising: (i) contacting an immune cell with a test compound; and (ii) detecting a NPA response, wherein the NPA response is selected from the group consisting of: an increase in phase II enzyme expression; an increase in phase II enzyme activity; a reduction in the level of reactive oxygen species; an increase in the GSH:GSSH ratio; an increase in T_{II}1 response; an increase in contact hypersensitivity; and an increase in antigen presenting cell activity, and wherein the NPA response indicates the presence of an NPA.

In some embodiments the immune cell is a dendritic cell. In some embodiments, the immune cell is a T_{H}1 cell. In some embodiments, the immune cell is in redox disequilibrium. In some embodiments, the T_{H}1 response is an increase in T_{H}1 cytokine production, e.g., IL-12 or IFN-γ. In some embodiments, the T_{H}1 response is an increase in T_{H}1 related gene expression.

In some embodiments, the cell is from an aged animal or human. In some embodiments, the immune cell is a cell line or derived from a cell line. In some embodiments, the detecting step is in vitro. In some embodiments, the detecting step is in vivo. In some embodiments, the method further comprises a step of exposing the immune cell to an antigen. In some embodiments, the method further comprises inducing a state of redox disequilibrium in the immune cell.

### BRIEF DESCRIPTION OF THE DRAWINGS

FIG. 1. SFN reverses the age-related decrease in the CHS response. A. Ear-swelling response (mean±SD). B. Hematoxylin and eosin staining or ear tissue. C and D. Real-time PCR for mRNA levels of genes in the ear (FIG. 1, C) and liver (FIG. 1, D). Results represent the fold increase (means±SDs) compared with the CON-Young group (n=6), *P<0.05. **P<0.01, and ***P<0.001. CON=Vehicle-treated control; SFN=SFN treated; DFNB=DNFB sensitized/challenged.

FIG. 2. Nrf2 deficiency suppresses the CHS response. A. Ear-swelling response (means±SDs). B. IFN-γ and IL-4 mRNA levels were measured by means of real-time PCR. Results represent the fold increase (means±SDs) compared with the CON-Nrf2^{+/+} group (n=6), *P<0.05, **P<0.01, and ***P<0.001. CON=Vehicle-treated control; OXA=OXA sensitized/challenged.

FIG. 3. DCs from old mice have lower thiol levels and phase II mRNA expression. A. Mean fluorescence intensity for MBB staining in CD11c^{|} splenocytes from young and old mice (means±SDs). B. Phase II mRNA expression in CD11c⁺ splenocytes from young and old mice. Results represent the fold increase (means±SDs) of old compared with young mice (n=4), *P<0.05.

FIG. 4. DC redox disequilibrium interferes in the delayed-type hypersensitivity response on adoptive transfer. The car-swelling response in recipient mice (3 months old) receiving DNBS-pulsed DCs from young versus old mice (A) or nrf2^{+/+} versus nrf2^{-/-} mice (B), followed by DNFB challenge, is shown (n=6). *P<0.05, **P<0.01, and ***P<0.001. CON=Vehicle-treated control; DNBS=DNBS pulsed.

FIG. 5. N-aceytlcysteine (NAC) or SFN treatment or DCs reverses the age-related decrease in the CHS response on adoptive transfer. NAC-treated (A) or SFN-treated (B) DCs were exposed to DNBS ex vive and adoptively transferred into mice that were challenged with DNFB (mean±SD, n=6). *P<0.05, **P<0.01, and ***P<0.001. CON=Vehicle-treated control; DNBS=DNBS pulsed.

FIG. 6. Nrf2 deficiency suppresses the CHS response induced by DNFB. The ear-swelling response was expressed as mean±SD (n=4). *P<0.05, **P<0.01, and 20***P<0.001. CON=Vehicle-treated control; DNFB=DNFB sensitized/challenged.

FIG. 7. Thiol levels in the BM-DCs decreased by aging, Nrf2 deficiency, or both. Cultured BM-DCs were surface stained with phycoerythrin-labeled anti-CD 11c, followed by MBB staining and flow cytometry. A. Mean fluorescence intensity for MBB staining in CD11c⁺ splenocytes from young and old mice (means±SDs). B. Mean fluorescence intensity for MBB staining in CD11c⁺ splenocytes from old Nrf2^{+/+} and Nrf2^{-/-} mice (means±SD, n=4). *P<0.05. MFI=Mean fluorescence intensity.

FIG. 8. Increased thiol levels in the BM-DCs by NAC treatment. Cultured BM-DCs were incubated with or without NAC (20 mmol/L for 24 hours) and then washed. Cells were surface stained with phycoerythrin-labeled anti-CD 11c, followed by MBB staining and flow cytometry. The graph shows mean fluorescence intensity for MBB staining in CD11c⁺ BM-DCs (means±SDs, n−4). *P<0.05. CON—Control group.

FIG. 9. SFN treatment upregulates p2E message and thiol levels. Cultured BM-DCs were incubated with or without SFN (5 μmol/L for 24 hours) and then washed. A. Total RNA was extracted from CD11c⁺ BM-DCs to perform real-time PCR for p2E. B. Cells were surface stained with phycoerythrin-labeled anti-CD11c, followed by MBB staining and flow cytometry. The graph shows mean fluorescence intensity for MBB staining in CD11c⁺ BM-DCs (means t SDs, n=4). *P<0.05, ***P<0.001. CON=Control group; HO1=heme oxygenase 1.

### DETAILED DESCRIPTION OF THE INVENTION

### Introduction

The present invention focuses on reversing age-related impairments to redox equilibrium. Redox equilibrium is particularly restored to dendritic cells (DC), a primary antigen presenting cell (APC) in aging individuals. Sulforaphane (SFN), a naturally occurring chemical found in broccoli, activates Nrf2-mediated antioxidant phase II enzymes, restores redox equilibrium, and reverses age-related decline in APC activity of DC. The age-related decline in cellular immune function is thereby reversed. This effect is demonstrated in animals receiving SFN orally and those receiving treatment with DC treated ex vivo with SFN.

The present invention provides a number of advantages: (i) activation of the Nrf2 phase II enzyme pathway is surprisingly effective for inducing antioxidant activity; (ii) broccoli and other NPA-containing foods are safe and commercially-available; (iii) broccoli other NPA-containing foods are a relatively inexpensive source for nutraceutical development; (iv) oral administration; and (v) commercial availability of a powdered form.

The compositions of the invention can thus be applied as nutraceutical supplements to boost immune function in the elderly, and can be also be used to improve the efficacy of vaccines, e.g., the flu vaccine. The compositions of the invention can be used as stand-alone supplements, treatments, or to boost vaccine function, e.g., by co-administering SFN as an adjuvant with a vaccine. In some embodiments, SFN-treated DCs can be administered with the vaccine.

In some embodiments, a SFN composition or other composition that activates the Nrf2-mediated antioxidant pathway (NPA) is administered orally. The composition can include broccoli or another cruciferous vegetable rich in SFN or glucoraphenin, or an extract thereof.

The invention is based in part on the demonstration that the Nrf2 pathway affects contact hypersensitivity (CHS) and T_{H}1-mediated immune responses in old mice. Similar observations were made in vivo using ex vivo techniques to modify DC redox status. The results demonstrate that oral administration of a potent Nrf2 agonist, SFN, reverses the age-related decline of CHS responses. This effect can be reproduced when antigen-pulsed DCs from old mice are treated with SFN ex vivo before adoptive transfer. This finding is compatible with decreased Nrf2 expression, decreased p2E expression, and lower thiol levels in DCs from old animals. SFN and NAC restored the DC redox equilibrium, allowing DCs from old animals to function normally in vivo. The results described herein show that the state of redox equilibrium of DCs is important in the decrease of T_{H}1 immunity with aging

The present demonstration that redox disequilibrium in immune cells is related to age-related functional decline shifts the free radical theory of aging to an adaptive multifactorial process that is determined by a dynamic interplay between pro-oxidant and antioxidant forces (Lane, J Theor Biol, 225:531-40 (2003)). Thus, persons with decreased antioxidant protection can be more prone to immune senescence, and that dietary or therapeutic antioxidant intervention can reverse the injurious effects of oxidative stress in the immune system. Our data clearly show that it is possible to reverse the age-related decrease in T_{H}1 immunity in old mice within days of restoring redox equilibrium in the immune system.

The role of DCs in immune senescence has not been extensively reported. Moreover, previous studies have yielded conflicting results (Lung et al., Vaccine, 18:1606-12 (2000); Miller et al., Aging Immunol Infect Dis, 5:249-57 (1994); Pawelec et al., J Leukoc Biol, 64:703-12 (1998); Shurin et al., Crit Rev Oncol Hematol, 64:90-105 (2007)). For instance, some studies did not observe a difference in DC surface marker expression, whereas others have shown decreased MHC class II and costimulatory receptor expression in aged individuals. Other age-related abnormalities reported for DCs with aging include impaired recruitment, decreased transportation of antigens to lymph node germinal centers, impaired IFN-γ production, and interference in APC activity caused by a putative increase in IL-10 levels.

The present disclosure provides the first demonstration that DCs from older animals exhibit lower thiol levels and decreased expression of Nrf2 and p2E message levels. The present results rely on myeloid DCs, which are derived from the same precursors as Langerhans Cells (LCs). LCs are notoriously difficult to isolate in significant numbers. The present results provide proof of principle that DCs from old animals do not perform as well as DCs from young animals in the adoptive transfer model. The procedures and results are highly reproducible, and allowed us to show that manipulation of DC redox status influences APC activity in vivo. Nrf2 activity exerts major effects on APC function, which is directly relevant to the study of aging because Nrf2 levels are reduced in the elderly.

In spite of the decrease in Nrf2 activity with aging, the present disclosure demonstrates that SFN can effectively restore redox equilibrium in old animals in parallel with an improvement in CHS and T_{H}1 immunity. Thus, broccoli and other cruciferous vegetables containing SFN, glucosinolate, or other NPAs can be used to improve immune function in the elderly. In addition, the electrophilic chemistry that leads to Nrf2 release from its chaperone provides a platform for further drug discovery. Further, treatment of old rats with α-lipoic acid can increase nuclear Nrf2 levels in parallel with increased γ-CLC expression and GSH production. Finally, we demonstrate the potential for using DCs to conduct vaccination therapy as a means of restoring in vivo cellular immune function during aging.

### DEFINITIONS

As used herein, a “Nrf2 pathway agonist” (NPA) includes any substance that increases intracellular antioxidant activity, e.g., by increasing expression or activity of Phase II enzymes (p2Es), glutathione (GSH), glutathione peroxidase (GPx), γ-glutamylcysteine ligase (γ-GCL), hemeoxygenase 1, superoxide dismutase, glutathione S transferase, and reduced nicotinamide adenine dinucleotide phosphate quinine oxidoreductase (NQO1). A non-limiting list of NPAs includes: sulforaphane (SFN), compounds involved in sulforaphane synthesis (e.g., glucoraphanin), α-lipoic acid, and SFN-containing substances. SFN or glucoraphanin can be found in cruciferous vegetables, including broccoli, Brussels sprouts, cabbage, cauliflower, bok choy, kale, collards, broccoli sprouts, Chinese broccoli, radish, rocket, and watercress. NPAs can also include other signaling molecules that upregulate expression of p2Es in a manner similar to Nrf2. NPAs include deliverable expression constructs comprising the polynucleotide sequences encoding NPA polypeptides, e.g., coding sequences for Nrf2 and p2Es.

The term “nutraceutical” generally refers to a food or food extract that has a medicinal effect on human health. The nutraceutical can be contained in a medicinal format such as a capsule, tablet, or powder in a prescribed dose.

In the context of the present invention, an “individual in need thereof” refers to an individual with reduced immune function as a result of age and/or age-related redox disequilibrium in immune cells. The decline in immune system function is gradual and varies by individual, thus, there is no precise age at which all individuals are definitively in need of treatment according to the invention. In some embodiments, an individual in need thereof is “aged,” or over 30, 40, 50, or over 60 years old. However, the compositions of the invention are advantageously applied earlier, especially in individuals with compromised immune systems. An individual in need thereof can also include individuals at risk of reduced immune function. e.g., an aging individual that does not necessarily exhibit a decline in immune function.

An individual in need thereof can also be an individual at an increased risk of infection, e.g., an individual that will undergo medical treatment or surgery, travel to an unfamiliar environment, or be exposed to an unfamiliar population.

As used herein, an “immunocompromised” individual refers to an individual with reduced immune function resulting from age, medical treatment (e.g., chemotherapy or other drug-related side effect), disease, or infection (e.g., HIV). Immunocompromised individuals thus include those with primary (e.g., hereditary) and acquired immunodeficiencies. Diagnosis of an immunodeficiency is within the skill in the medical arts.

As used herein, “improving immune function” refers to any increase in immune function, including. The term “improvement” or “increase” are relative, e.g., as compared to a control. Selection of appropriate controls is well within the skill in the art, and depends on the particular patient circumstances or purpose of the investigation. For example, in some embodiments, an improvement in the measured immune function is observed relative to that immune function prior to treatment in the same individual. In some embodiments, the improvement is observed compared to a different, but similar individual or group of similar individuals. In some embodiments, the improvement is observed compared to an average value or level for the particular immune function being measured gathered from a population of individuals.

The term “improving the efficacy of a vaccine” refers to any increase in the efficacy of a vaccine. For example, the vaccine can prevent or reduce the severity of the infection targeted by the vaccine, or improve the immune response of the individual to the vaccine antigen, even if the infection is not entirely prevented. Again, the terms “improve” and “increase” are relative, as will be understood in the art. For example, a composition of the invention can improve the efficacy of a vaccine relative to the efficacy of the same or related vaccine administered to the individual in the absence of the composition. In some embodiments, the improved efficacy is determined relative to an average efficacy gathered from a population of similar individuals, e.g., in the same age range.

The terms “effective amount or dose” or “sufficient amount or dose” refer to a dose that produces effects for which it is administered, e.g., improving immune function and vaccine efficacy. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

An “adjuvant” is a substance that stimulates the immune system and increase the response to a vaccine, without having antigen specificity in itself. Common examples are oils and aluminum salts. An NPA can also be used as an adjuvant.

The “innate immune system” includes the cells and processes that provide non-specific protection to its host. The cells of the innate system recognize and respond to pathogens (e.g., bacteria, fungal agents, and viruses) in a generic way, but do not provide a long-lasting response or protection. The innate immune system includes inflammatory cells (e.g., macrophages, dendritic cells, neutrophils, natural killer cells, mast cells, eosinophils, etc.) and components of the complement cascade. Cells involved in the innate immune system can act as antigen presenting cells and secrete cytokines to activate the adaptive immune system (T cells and B cells).

The “adaptive immune system” is antigen specific, and can confer lasting protection against a given pathogen or antigen. Adaptive immune cells include T cells and B cells. T cells are divided into cytotoxic T cells and helper T cells, which are in turn categorized as T_{H}1 or T_{H}2 helper T cells.

A “T_{H}1 response” is characterized by the production of IFN-g, which activates macrophages, and induces B-cells to make opsonizing antibodies. The T_{H}1 response is often referred to as cell-mediated immunity, and is generally effective against intracellular pathogens.

### Nrf2 and the Nrf2 Pathway

Nrf2 is a member of the CNC family of bZIP transcription factors. Nrf2 dimerizes with other family members and regulates gene expression through the antioxidant response element (ARE). Expression of phase II enzymes (p2Es) is controlled by the ARE. P2Es include glutathione (GSH), glutathione peroxidase (GPx), γ-glutamylcysteine ligase (γ-GCL), hemeoxygenase 1, superoxide dismutase, glutathione S transferase, and reduced nicotinamide adenine dinucleotide phosphate quinine oxidoreductase (NQO1).

When bound to its chaperone, Keap1, Nrf2 has a relatively short half-life (<20 minutes) and is continuously being degraded by a ubiquitin-26S proteosome pathway. Keap1 expresses 25 free cysteine residues, among which Cys-151 is critical for the binding and sequestration of Nrf2 (Dinkova-Kostova et al., Proc Natl Acad Sci USA, 99:11908-13 (2002)). Aging can affect the oxidation status and function of this thiol group. Moreover, Nrf2 autoregulates its own gene expression, which means that a decrease in ARE activity could have detrimental effects on the expression of this transcription factor (Kwak et al., Mol Cell Biol, 22:2983-92 (2002)). This age-related decrease in ARE transcriptional activity is also influenced by transcription factors that heterodimerize with Nrf2. Nrf2 binds to other bZip proteins, including members of the Jun/Fos family, Fra, small Maf, and ATF4 proteins (Itoh, K. et al., Biochem Biophys Res Common, 236:313-22 (1997)). Aging affects the transcriptional activity of a number of these binding partners.

Nrf2 protects memory T cells from age-related oxidant injury, including protection against the decrease in mitochondrial function and phenotypic changes in the T cell compartment with aging (Kim and Nel, J Immunol, 175:2948-59 (2005)). Nrf2 knockout mice have exaggerated cytokine production by innate cellular elements (Thimmulappa et al., J Clin Invest, 116:984-95 (2006)). Nrf2 also regulates the antigen-presenting cell (APC) activity of dendritic cells (DCs). Exposure of myeloid DCs to exogenous oxidative stress stimuli (e.g., pro-oxidative chemicals) interferes in IL-12 production and T_{H}1 immunity (Chan et al., J Allergy Clin Immunol, 118:455-65 (2006)). There is also growing evidence that the opposite might be true, namely that boosting of GSH levels at the APC level might favor T_{H}1 skewing of the immune response (Peterson et al., Proc Natl Acad Sci USA, 95:3071-6 (1998); Kim, et al., J Allergy Clin Immunol, 119:1225-3 (2007)).

Because GSH is such a potent antioxidant, small changes in GSH content can lead to sizeable biologic effects. Even a small decrease in GSH content can lead to a big decrease in the GSH/GSSG ratio. The GSH/GSSG ratio under conditions of redox equilibrium is typically in the range of 40:1 to 50:1.

A non-limiting list of NPAs includes: sulforaphane (SFN), agents involved in sulforaphane synthesis (e.g., myrosinase and glucoraphanin), α-lipoic acid, and substances that contain these compounds. SFN or glucoraphanin can be found in cruciferous vegetables, including broccoli, Brussels sprouts, cabbage, cauliflower, bok choy, kale, collards, broccoli sprouts, Chinese broccoli, radish, rocket, and watercress. An NPA can also include other transcription factors that affect expression of p2Es similar to Nrf2.

### Methods of Screening for Nrf2 Pathway Agonists

The present invention provides methods of screening for agents for improving immune function or improving the efficacy of a vaccine. Such agents target and activate the Nrf2 antioxidant pathway.

Nrf2 pathway agonists (NPAs) include agents that increase intracellular antioxidant activity, e.g., by increasing expression, cellular concentration, or activity of a Phase II enzyme (p2E), glutathione (GSH), glutathione peroxidase (GPx), γ-glutamylcysteine ligase (γ-GCL), hemeoxygenase 1, superoxide dismutase, glutathione S transferase, and reduced nicotinamide adenine dinucleotide phosphate quinine oxidoreductase (NQO1). Nrf2 pathway agonists also include agents that inhibit the ability of KEAP, a Nrf2 chaperone protein, to bind to Nrf2 and target it for degradation. A non-limiting list of NPAs includes: sulforaphane (SFN), agents involved in sulforaphane synthesis (e.g., myrosinase and glucoraphanin), α-lipoic acid, and SFN-containing substances. SFN or glucoraphanin can be found in cruciferous vegetables, including broccoli, Brussels sprouts, cabbage, cauliflower, bok choy, kale, collards, broccoli sprouts, Chinese broccoli, radish, rocket, and watercress. NPAs can also include other signaling molecules that upregulate expression of p2Es in a manner similar to Nrf2. NPAs include deliverable expression constructs comprising the polynucleotide sequences encoding NPA polypeptides, e.g., coding sequences for Nrf2 and p2E.

Agents to be identified through the present screening methods can be any compound or composition. Furthermore, the test agent exposed to a cell or protein according to the screening methods of the present invention can be a single compound or a pool of compounds. When a pool of compounds is contacted with Nrf2, a Nrf2 expressing cell, or an immune cells, the compounds can be contacted sequentially or simultaneously. For example, a pool of test agents can be applied to a plurality of immune cells to determine if any of the test agents within the pool modulate Nrf2 pathway antioxidant activity. If there is a change in activity, then the pool of test agents can be narrowed down until the effective NPA is identified. Thus, in some embodiments, a single test agent is contacted with Nrf2, a Nrf2 expressing cell, or an immune cell in a single sample, e.g., using multiwell plates, or an array. In some embodiments, a plurality of test agents is contacted with Nrf2, a Nrf2 expressing cell, or an immune cell in a single sample.

### Test Agents

Any test agent can be used in the screening methods of the present invention, for example, antibodies and antigen-binding fragments thereof, cell extracts, cell culture supernatant, products of fermenting microorganism, extracts from marine organism, plant extracts, purified or crude proteins, peptides, non-peptide compounds, synthetic micromolecular compounds, and nucleic acid constructs, such as antisense RNA, siRNA, ribozymes, etc. In some embodiments, the test agents are modified versions of sulforaphane. In some embodiments, the test agents are obtained from an electrophile library.

The test agent of the present invention can be also obtained using any of the numerous approaches in combinatorial library methods known in the art, including
(1) biological libraries,
(2) spatially addressable parallel solid phase or solution phase libraries,
(3) synthetic library methods requiring deconvolution,
(4) the “one-bead one-compound” library method and
(5) synthetic library methods using affinity chromatography selection.

The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Sci USA 1993, 90: 6909-13; Erb et al., Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Carell et al., Angew Chem Int Ed Engl 1994, 33: 2061; Gallop et al., J Med Chem 1994, 37: 1233-51). Libraries of compounds can be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698, 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6: Cwirla et al., Proc Natl Acad Sci USA 1990, 87: 6378-82; Felici, J Mol Biol 1991, 222: 301-10; US Pat. Application 2002103360).

The test agent can be also obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam, Anticancer Drug Des 1997, 12: 145-67). Examples of methods for the synthesis of molecular libraries can be found in the art (DeWitt et al., Proc Natl Acad Set USA 1993, 90: 6909-13; Erb et al, Proc Natl Acad Sci USA 1994, 91: 11422-6; Zuckermann et al., J Med Chem 37: 2678-85, 1994; Cho et al., Science 1993, 261: 1303-5; Carell et al., Angew Chem Int Ed Engl 1994, 33: 2059; Gallop et al. J Med Chem 1994, 37: 1233-51). Libraries of compounds can be presented in solution (see Houghten, Bio/Techniques 1992, 13: 412-21) or on beads (Lam, Nature 1991, 354: 82-4), chips (Fodor, Nature 1993, 364: 555-6), bacteria (U.S. Pat. No. 5,223,409), spores (U.S. Pat. Nos. 5,571,698; 5,403,484, and 5,223,409), plasmids (Cull et al., Proc Natl Acad Sci USA 1992, 89: 1865-9) or phage (Scott and Smith, Science 1990, 249: 386-90; Devlin, Science 1990, 249: 404-6; Cwirla et al., Proc Natl Acad Sci USA 1990, 87: 6378-82; Felici, J Mol Biol 1991, 222: 301-10; US Pat. Application 2002103360).

### Cell Free Assays

The invention provides methods of identifying an agonist of the Nrf2 pathway in a cell free assay. In some embodiments, the screening method comprises contacting test compounds with various domains of Nrf2, e.g., attached to a solid substrate, in order to determine if and where the test agent binds Nrf2. Exemplary domains of Nrf2 include the dimerization domain, the leucine zipper, the C terminal coiled coil, the DNA binding domain, the KEAP binding domain, and the domains involved in binding ATF4, Jun, CrebBP, etc. In addition to Nrf2, other Nrf2 antioxidant pathway members, or fragments thereof, can be used for the present screening.

Thus, the invention further provides methods of identifying a Nrf2 pathway agonist (NPA), said method comprising: (i) contacting Nrf2 or a Nrf2 pathway member with a test compound; and (ii) detecting interaction between the test agent and Nrf2 or the Nrf2 pathway member, wherein an interaction indicates the presence of an NPA.

The binding of a test agent to Nrf2 can be, for example, detected by immunoprecipitation using an antibody against the polypeptide, e.g., as described herein. Alternatively, the Nrf2 polypeptide or a fragment thereof can be expressed as a fusion protein with a known tag, e.g., polyhistidine, HA, β-galactosidase, maltose binding protein, glutathione S-transferase, green florescence protein (GFP). Vectors encoding such tags, and including multiple cloning sites are commercially available and can be used for the present invention.

For immunoprecipitation, an immune complex is formed by adding an antibody (recognizing Nrf2 or a tag) to a reaction mixture of Nrf2 and the test agent(s). Binding ability of a test agent to Nrf2 can be examined by, for example, measuring the size of the formed immune complex. Any method for detecting the size of a substance can be used, including chromatography, electrophoresis, and such. For example, when mouse IgG antibody is used for the detection, Protein A or Protein G sepharose can be used for quantitating the formed immune complex. For more details on immunoprecipitation see, for example, Harlow et al., Antibodies, Cold Spring Harbor Laboratory publications, New York, 1988, 511-52.

The Nrf2 polypeptide or fragment thereof can also be bound to a carrier. Example of carriers that can be used for binding the polypeptides include insoluble polysaccharides, such as agarose, cellulose and dextran; and synthetic resins, such as polyacrylamide, polystyrene and silicon; or commercially available beads and plates (e.g., multi-well plates, biosensor chip, etc.) prepared from the above materials can be used. Magnetic beads can also be used. The binding of a polypeptide to a carrier can be conducted according to routine methods, such as chemical bonding and physical adsorption. Binding of a polypeptide to a carrier can also be conducted by means of interacting molecules, such as the combination of avidin and biotin.

Screening using such carrier-bound Nrf2 can comprise the steps of contacting a test agent to the carrier-bound polypeptide, incubating the mixture, washing the carrier, and detecting and/or measuring the test agent bound to the carrier. The binding can be carried out in buffer, e.g., phosphate buffer or Tris buffer, as long as the buffer does not inhibit the binding.

### Cell-Based Assays

The invention includes cell-based assays to screen for agonists of the Nrf2 pathway. Such methods can be carried out in immune cells, such as T_{H}1 cells and dendritic cells. Nrf2 activity can be determined according to methods common in the art and described herein.

Thus the invention provides methods of identifying a Nrf2 pathway agonist (NPA), said method comprising: (i) contacting an immune cell with a test compound; and (ii) detecting a NPA response, wherein the NPA response is selected from the group consisting of: an increase in phase II enzyme expression; an increase in phase II enzyme activity; a reduction in the level of reactive oxygen species; an increase in the GSH:GSSH ratio; an increase in T_{H}1 response; an increase in contact hypersensitivity; and an increase in antigen presenting cell activity, and wherein the NPA response indicates the presence of an NPA.

In some embodiments, the immune cell is in redox disequilibrium. In some embodiments, the T_{H}1 response is an increase in T_{H}1 cytokine production, e.g., IL-12 or IFN-γ. In some embodiments, the T_{H}1 response is an increase in T_{H}1 related gene expression.

In some embodiments, the cell is from an aged animal or human. In some embodiments, the immune cell is a cell line or derived from a cell line. In some embodiments, the detecting step is in vitro. In some embodiments, the detecting step is in vivo. In some embodiments, the method further comprises a step of exposing the immune cell to an antigen. In some embodiments, the method further comprises inducing a state of redox disequilibrium in the immune cell.

### High-Throughput Assays

In some embodiments, the assays are designed to screen large chemical libraries by automating the assay steps and providing compounds from any convenient source to assays, which are typically run in parallel (e.g., in microtiter formats on microtiter plates in robotic assays). One or more negative control reactions that do not include a test agent or modulator are included in the assay system. It is also desirable to have positive controls to ensure that the components of the assays are working properly. For example, a known NPA (e.g., SFN) can be incubated with one sample of the assay, and the resulting change in activity is then determined according to the methods described herein.

In the high throughput assays of the invention, it is possible to screen up to several thousand different candidate compounds in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (96) compounds. If 1536 well plates are used, then a single plate can easily assay from about 100-about 1500 different compounds. It is possible to assay many different plates per day; assay screens for up to about 6,000-20,000, and even up to about 100,000-1,000,000 different compounds is possible using the integrated systems of the invention.

### Nutraceutical and Pharmaceutical Compositions and Administration

The agents as described herein (e.g., NPAs) can be administered to a human patient in accord with known methods. Information regarding pharmaceutical formulation and administration are detailed in Remington: The Science and Practice of Pharmacy, Gennaro, ed., Mack Publishing Co., Easton, Pa., 19th ed., 1995.

In its simplest forms, the NPAs of the invention can be administered as whole foods. NPA-containing foods include cruciferous vegetables, as listed above. Extracts that are particularly concentrated for, e.g., SFN, can also be prepared.

The compositions can be administered for therapeutic or prophylactic treatments. In therapeutic applications, compositions are administered to an individual in need thereof (e.g., an aged or immunocompromised individual) in a “therapeutically effective dose.” Amounts effective for this use will depend upon the mode of administration (e.g., oral, topical, parenteral, intravenous), the general state of the patient's health, and the patient's age, weight, and pharmacological profile. Single or multiple administrations of the compositions can be administered depending on the dosage and frequency as required and tolerated by the patient. An individual, patient, or subject, for the purposes of the present invention includes both humans and other animals, particularly mammals. Thus the methods are applicable to both human therapy and veterinary applications.

The pharmaceutical compositions can be administered in a variety of unit dosage forms depending upon the method of administration. For example, unit dosage forms suitable for oral administration include, but are not limited to, powder, tablets, pills, capsules and lozenges. In some cases, e.g., with combination therapies, oral administration requires protection from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art. Compositions for topical administration are also included, e.g., creams, powders (e.g., to be rehydrated), gels, sprays, etc.

Pharmaceutical formulations of the present invention can be prepared by mixing an agent having the desired degree of purity with optional pharmaceutically acceptable carriers, excipients or stabilizers. Such formulations can be lyophilized formulations or aqueous solutions. Acceptable carriers, excipients, or stabilizers are nontoxic to recipients at the dosages and concentrations used. Acceptable carriers, excipients or stabilizers can be acetate, phosphate, citrate, and other organic acids; antioxidants (e.g., ascorbic acid), preservatives, low molecular weight polypeptides; proteins, such as serum albumin or gelatin, or hydrophilic polymers such as polyvinylpyllolidone; and amino acids, monosaccharides, disaccharides, and other carbohydrates including glucose, mannose, or dextrins; chelating agents; and ionic and non-ionic surfactants (e.g., polysorbate); salt-forming counter-ions such as sodium; metal complexes (e.g. Zn-protein complexes); and/or non-ionic surfactants.

The formulation can also provide additional active compounds, including in particular a vaccine, or an adjuvant. The active ingredients can also prepared as sustained-release preparations (e.g., semi-permeable matrices of solid hydrophobic polymers (e.g., polyesters, hydrogels (for example, poly (2-hydroxyethyl-methacrylate), or poly (vinylalcohol)), polylactides. Vaccine antigens and/or adjuvants can also be entrapped in microcapsules prepared, for example, by coacervation techniques or by interfacial polymerization, for example, hydroxymethylcellulose or gelatin microcapsules and poly-(methylmethacylate) microcapsules, respectively, in colloidal drug delivery systems (for example, liposomes, albumin microspheres, microemulsions, nano-particles and nanocapsules) or in macroemulsions.

The compositions for administration will commonly comprise an Nrf2 pathway agonist (NPA) dissolved in a pharmaceutically acceptable carrier, preferably an aqueous carrier. A variety of aqueous carriers can be used, e.g., buffered saline and the like. These solutions are sterile and generally free of undesirable matter. These compositions can be sterilized by conventional, well known sterilization techniques. The compositions can contain pharmaceutically acceptable auxiliary substances as required to approximate physiological conditions such as pH adjusting and buffering agents, toxicity adjusting agents, for example, sodium acetate, sodium chloride, potassium chloride, calcium chloride, sodium lactate and the like. The concentration of active agents in these formulations can vary widely, and will be selected primarily based on fluid volumes, viscosities, body weight and the like in accordance with the particular mode of administration selected and the patient's needs.

In the case of vaccines, aqueous solutions are commonly administered by injection, e.g., intravenous administration, as a bolus or by continuous infusion over a period of time. Alternatively administration can be intramuscular, intraperitoneal, intracerobrospinal, subcutaneous, intra-articular, intrasynovial, intrathecal, oral, topical, or inhalation routes. Intravenous or subcutaneous administration of the antibody is preferred. The administration can be local or systemic

Thus, a typical pharmaceutical composition for intravenous administration will vary according to the agent. Actual methods for preparing parenterally administrable compositions will be known or apparent to those skilled in the art and are described in more detail in such publications as Remington's Pharmaceutical Science, 15th ed., Mack Publishing Company. Easton, Pa. (1980).

The pharmaceutical preparation is preferably in unit dosage form. In such form the preparation is subdivided into unit doses containing appropriate quantities of the active component(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

### Combination Therapies

The agents disclosed herein can be effectively combined with vaccines and other immunotherapeutic agents. Such combination therapies are helpful for reducing the dose of each individual component and reducing unwanted side effects. Combination therapy is particularly helpful in the case of aged and elderly individuals, where vaccination frequently fails. The nutraceuticals of the invention improve immune function in immunocompromised and aged individuals, thereby increasing the likelihood that a vaccine will be effective.

Vaccines can be in combination or alone. Exemplary vaccines include those against; influenza, pneumonia, rabies, tetanus, shingles/chickenpox, pertussis, hepatitis (A, B, or C), HPV, polio, mumps, measles, rubella, diphtheria, HiB, rotavirus, and meningococcal disease.

In some embodiments, the nutraceuticals of the invention are advantageously combined with other immunotherapies, e.g., chemotherapies or drugs that reduce immune function. Such combinations can be helpful for reducing the decline in immune function that accompanies such therapies.

As an example, an NPA can be combined in the same composition with a vaccine or other therapeutic agent. In some embodiments, the NPA is actually joined to at least a functionally active portion of a vaccine or therapeutic agent.

In some cases, it is desirable to administer the therapeutic agents separately. In this case, the dose and frequency of administration of each component of the combination can more easily be controlled and varied.

One of skill will understand that one or more nutraceutical of the invention (e.g., NPA) can be combined with one or more vaccine or therapeutic agent.

Although specific embodiments of the invention have been described herein for purposes of illustration, various modifications can be made without deviating from the spirit and scope of the invention. Accordingly, the invention is not limited to the specific embodiments disclosed. All publications, patents, and patent applications cited herein are incorporated by reference in their entireties for all purposes.

### EXAMPLES

The dynamic equilibrium between the Nrf2 pathway and injurious oxidant stress responses can determine the effect of aging in the immune system. This is compatible with the tendency toward a generalized decrease in GSH levels and γ-GCL expression with aging. Aging also leads to a decrease in Nrf2 activity and Phase II Enzyme (p2E) expression in parallel with increased markers of oxidative stress. Although the exact reason for decreased Nrf2 activity is unknown, aging leads to decreased binding of this transcription factor to the antioxidant response element (ARE), which regulates the transcriptional activation of p2E gene promoters. The decrease in antioxidant activity is exaggerated during aging of female nrf2 knockout mice. In spite of this decrease in Nrf2 activity, p2E expression and GSH production in old rats is correctable by the Nrf2 agonist, α-lipoic acid. The fact that the Nrf2 pathway remains responsive in the aged is encouraging for related therapies using more potent agonists, such as the broccoli chemical sulforaphane (SFN).

The present examples demonstrate that SFN improves cellular immune responses in older mice using contact hypersensitivity (CHS). CHS is a commonly measured type of cellular immunity, and is known to decrease with aging.

### Materials and Methods

### Mice

Young (2-4 months) and old (19-22 mouths) female C57BL/6 (B6) mice were obtained from the Jackson Laboratory and the National Institute of Aging colony (Bethesda, Md.), respectively. Nrf2^{+/+} and nrf2^{-/-} mice, which were initially obtained from Dr. Y. Kan (Chan and Kan, Proc Natl Acad Sci USA, 96:1231-6 (1997)), were backcrossed onto a C57BL/6 background for 7 generations. Neither the CHS procedure nor SFN administration had any effects on the overall well-being or body weight of the animals.

### Reagents

RPMI-1640 and FCS were obtained from Cellgro (Herndon, Va.) and Irvine Scientific (Santa Ana, Calif.), respectively. OXA and DNFB were purchased from Sigma (St. Louis, Mo.). DNBS was obtained from MP Biomedicals, Inc (Irvine, Calif.). MBB was purchased from Molecular Probes (Eugene. Ore). Antibodies for cellular staining of CD11c were obtained from BD PharMingen (San Diego, Calif.). Primers for real-time PCR (see below) were purchased from E-Oligos (Hawthorne, N.Y.). All organic solvents were of Fisher Optima grade, and the solid chemicals were of analytic reagent grade.

CHS Testing with Contact-Sensitizing Agents

Oxazalone (OXA; 3%), dissolved in 100% ethanol, was applied on the shaved mouse abdomen on day 0. Control animals were exposed to vehicle alone. Six days after sensitization, mice were challenged on both sides of both ears by means of epicutaneous application of 20 μL of a 1% OXA solution (Gaspari and Katz, Current Protocols in immunology, John Wiley & Sons (Hoboken, N.J., 2003)). 2,4-Dinitro-1-fluambenzene (DNFB) sensitization was accomplished by the application of 0.5% of the chemical dissolved in 4:1 acetone/olive oil onto the shaved abdomen (days 0 and 1). On day 5, mice were challenged by means of epicutaneous application of 0.2% DNFB on both ears. Ear thickness was measured before and 24 and 48 hours after challenge by using a dial thickness gauge (Mitutoyo, Japan). Mice were killed 48 hours after challenge, and ear tissues were removed for RNA extraction and cytokine message expression, as well as for hematoxylin and eosin staining.

### SFN Oral Administration

SFN (9 μmol/d per mouse) in 0.2 mL, of corn oil was administered by means of gavage on consecutive days. The control group received corn oil alone. Pretreatment with SFN or corn oil commenced 5 days before and was carried through until the performance of the antigen challenge (i.e., 11 days total).

### RIVA Isolation

Total RNA was extracted from DCs or tissues by using the RNeasy Mini Kit (Qiagen, Inc, Valencia, Calif.), according to the manufacturer's recommendations. Contaminating DNA was removed with a DNA-free kit (Ambion, Inc, Austin, Tex.). Total RNA was spectrophotometrically quantitated. A 1 μg RNA sample was reverse transcribed by using the iScriptTMcDNA Synthesis Kit (Bio-Rad Laboratories, Hercules, Calif.). The cDNA templates were stored at −40° C.

### Real-Time RT-PCR

PCR was carried out with an iQTM SYBR Green Supermix (Bio-Rad Laboratories) by using an iCycler (Bio-Rad Laboratories), according to the manufacturer's instructions. The sequences for the primers for IFN-γ, IL-4, T-bet, heme oxygenase 1, Nrf2, NQO1, glutathione S-transferase, GCLS, GPx, and β-actin are summarized in Table 1. The final PCR mixture contained 1 μL of cDNA template and 400 nM of the forward and reverse primers in a final volume of 25 μL. Samples were run concurrently with a standard curve prepared from the PCR products. Serial dilutions were performed to obtain appropriate template concentrations. B-Actin was used as a reference gene for the recovery of RNA, as well as reverse transcription efficiency. Melting curve analysis was used to confirm specific replicon formation.

TABLE E1
The primer sequences for real-time PCR analysis

| Primers | Forward (5′-3′) | Reverse (5′-3′) |
|---|---|---|
| IFN-γ | ACTGGCAAAAGGATGGTGAC | TGAGCTCATTGAATGCTTGG |
| IL-4 | TCAACCCCCAGCTAGTTGTC | TGTTCTTCGTTGCTGTGAGG |
| T-bet | CAACAACCCCTTTGCCAAAG | TCCCCCAAGCAGTTGACAGT |
| HO-1 | CACGCATATACCCGCTACCT | CCAGAGTGTTCATTCGAGCA |
| Nrf2 | CTCGCTGGAAAAAGAAGTGG | CCGTCCAGGAGTTCAGAGAG |
| NQO1 | TTCTCTGGCCGATTCAGAGT | CCTGTTGCCCACAAGGTAGT |
| GST | CGCCACCAAATATGACCTCT | CCTGTTGCCCACAAGGTAGT |
| γ-GCLS | TGGAGCAGCTGTATCAGTGG | ATGAGCAGTTCTTTCGGGTCA |
| GPx | GTCCACCGTGTATGCCTTCT | TCTGCAGATCGTTCATCTCG |
| β-actin | AGCCATGTACGTAGCCATC | CTCTCAGCTGTGGTGGTGA |

### Generation of Bone Marrow-Derived DCs

Bone marrow-derived DCs (BM-DCs) were prepared as previously described (Kim, et al., J Allergy Clin Immunol, 119:1225-33 (2007)). Briefly, bone marrow cells were removed from the femurs of mice and cultured at a concentration of 2×10⁶ coils pet well in 6-well culture plates. Each well received 2 ml of RPMI-1640 supplemented with 10% FCS, 1% penicillin/streptomycin, 1% glutamine, 55 μmol/2-mercaptoethanol, GM-CSF (40 ng/mL), and IL-4 (100 pg/mL). The culture medium was refreshed every 3 days.

### Surface Staining, Monobromobimane Staining, and Flow Cytometry

Cells were surface stained with phycoerythrin-labeled anti-CD11c. Cells were incubated with antibodies for 30 minutes at 4° C. in staining buffer in the dark. Samples were analyzed in the LSR flow cytometer (BD PharMingen) by using the excitation and emission settings of 488 nm and 575 nm (FL-2 channel), respectively. A minimum of 20,000 events were collected and analyzed with CellQuest software (Becton Dickinson, San Jose, Calif.).

Monobromobimane (MBB) was used to stain intracellular thiol, followed by conducting flow cytometry, as previously described (Kim et al., J Allergy Clin Immunol, 119:1225-33 (2007)). Working solutions of MBB (1 mmol/L) in PBS were made fresh from a 40 mmol/L MBB stock solution in dimethyl sulfoxide. Cells were resuspended in PBS at a concentration of 10⁶ cells/mL, and MBB was added to a final concentration of 40 μmol/L for 10 minutes at room temperature. Where MBB fluorescence was combined with surface staining, this dye was added after surface staining, as described below. MBB fluorescence was excited by the UV laser tuned to 325 nm, and emission was measured at 510 nm (FL-4 channel) in the LSR flow cytometer.

Single-Cell Preparation from Spleens

The spleens were aseptically removed and gently grinded on a cell strainer in PBS. These single-cell suspensions were incubated with ammonium chloride to remove red blood cells. After washing with PBS, cells were resuspended in PBS.

Magnetic Bead Separation of CD11c⁺ Cells

Magnetic cell sorting was performed by using microbead-labeled anti-CD11c (Miltenyi Biotec, Bergisch Gladbach, Germany), as previously described (Kim and Nel, J Immunol, 175:2948-59 (2005); Chan et al., J Allergy Clin Immunol, 118:455-65 (2006)). Briefly, splenocytes were prepared as described and enriched with microbead-labeled anti-CD11c (Miltenyi Biotec). The labeled cells were separated by using the autoMACS (Miltenyi Biotec) system. The purity of the CD11c⁺ population was confirmed by means of flow cytometry.

### Eliciting CHS Responses by Means of Adoptive DC Transfer

CHS was induced by in vivo inoculation of antigen-pulsed DCs (Kim et al., J Allergy Clin Immunol. 119:1225-33 (2007)). Cultured BM-DCs were incubated with or without N-acetylcysteine (NAC) (20 mM for 1 hour) or SFN (5 μM for 24 hours) and then washed and resuspended in PBS containing 100 μg/mL 2,4-dinitrobenzene sulfonic acid (DNBS) for 30 minutes. For sensitization (day 0), 0.5×10⁶ DNBS-treated DCs were injected subcutaneously with 100 μL of saline into the flanks of recipient mice. Five days later, mice were challenged by means of DNFB application to the ear. Mice injected with the same number of unmodified DCs or mock treated and challenged with vehicle alone served as negative controls.

### Hematoxylin and Eosin Staining

The left ear from each killed animal was excised and fixed in 10% buffered formalin phosphate. After processing and staining with hematoxylin and cosin, the sections were examined in a Fisher Digital Micromaster I (Fisher Scientific, Hampton, N.H.) at a magnification of ×20. At least 10 fields were examined for each tissue section.

### Statistical Analysis

Results were expressed as means±SD and analyzed by using the Student t test. P values of less than 0.05 were considered significant.

### Results

### Example 1

### SFN Restores the Age-Related Decrease in the Contact Hypersensitivity (CHS) and T_{H}1 Immunity

Aging leads to a decrease of the CHS to contact antigens placed on the skin (Kim et al., J Allergy Clin Immunol, 119:1225-33 (2007)). Although a number of mechanisms might explain the increase in oxidant stress during aging, we considered the role of the Nrf2 pathway in the response outcome. Recent studies indicate that SFN significantly activates Nrf2-mediated phase II enzyme (p2E) gene expression that is absent in Nrf2-deficient animals. SFN administration can therefore be used to study the effect of the Nrf2 pathway on the decrease of T_{H}1 immunity in aging.

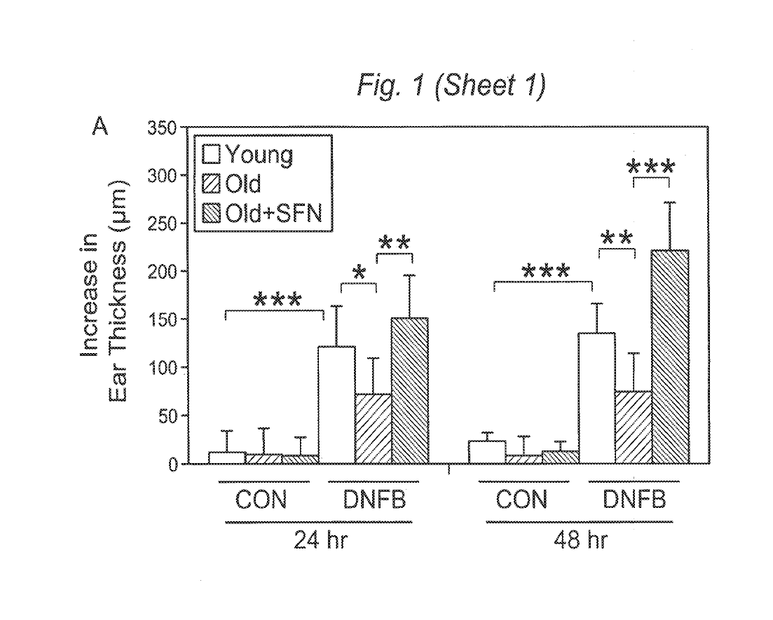

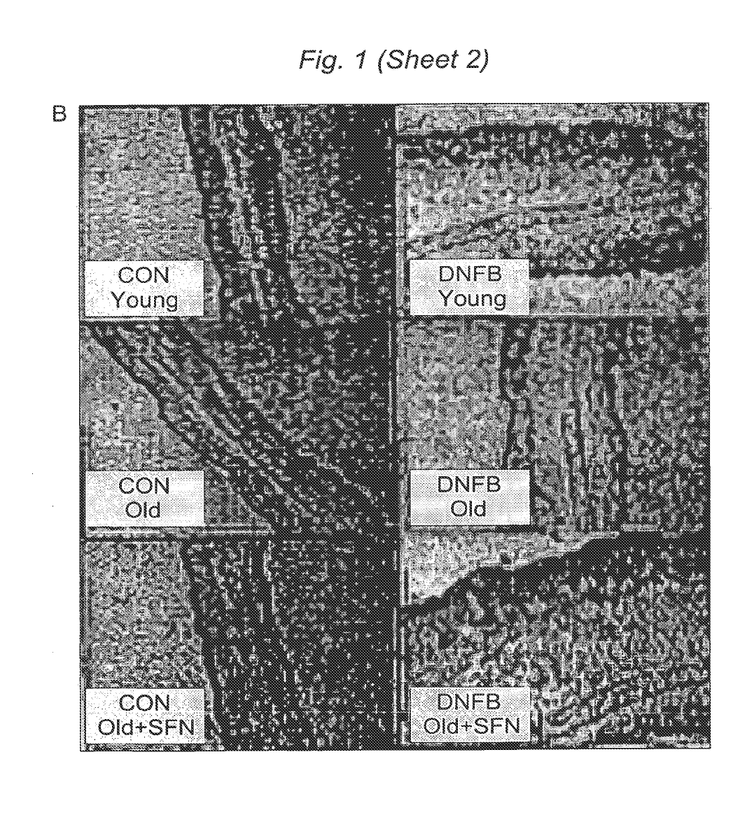

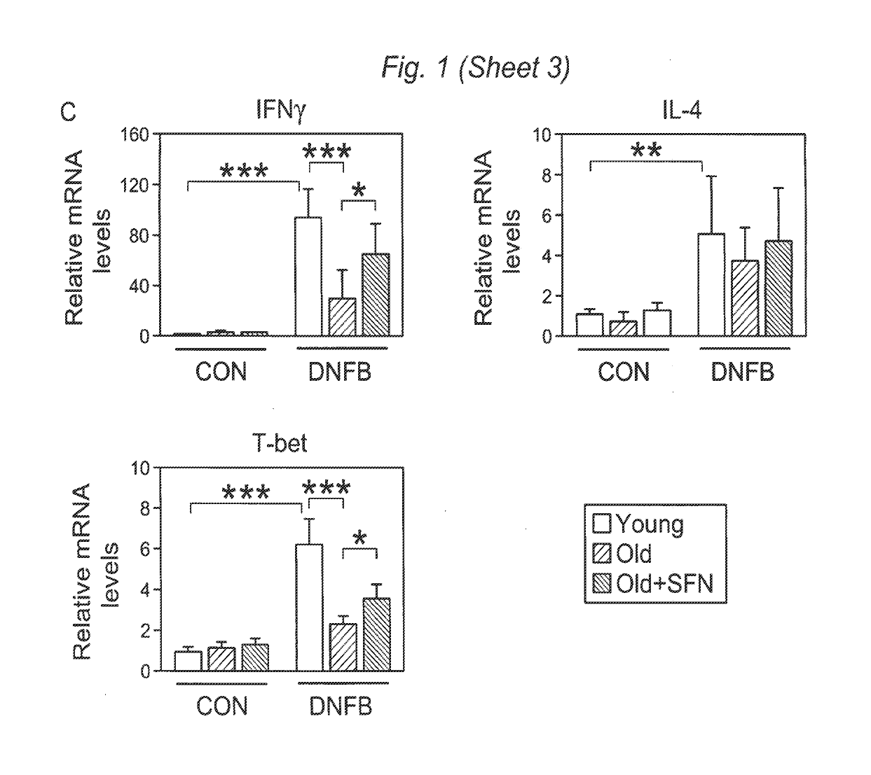

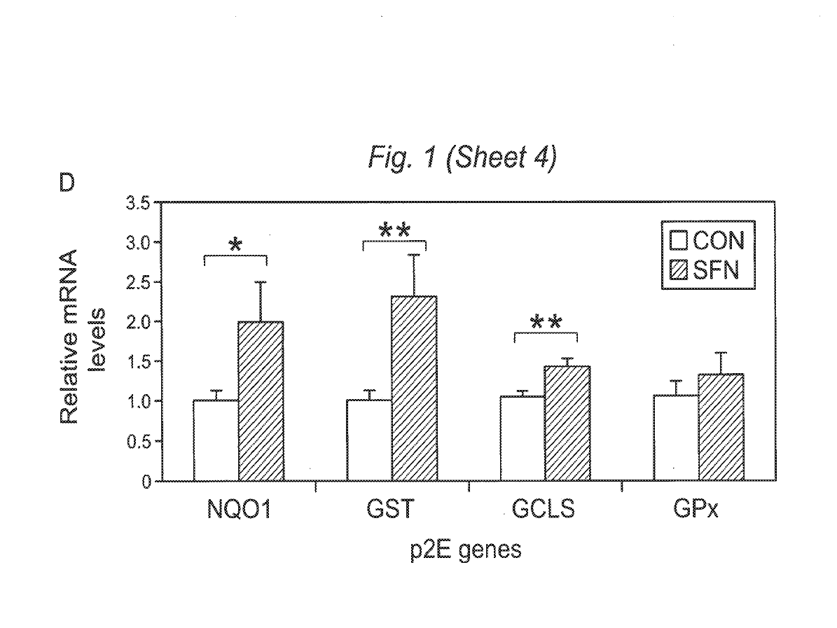

To determine whether SFN gavage affects the CHS response, a previously determined effective dose (9 μmol/d per mouse) of the nutraceutical was delivered to 20- to 22-month-old mice before performance of the ear-swelling responses (Thimmulappa et al., Cancer Res, 62:5196-203 (2002)). Nontreated animals of similar age or 2- to 3-month-old mice were used as comparative control animals. Indeed, the ear-swelling response to DNFB challenge was significantly reduced in old compared with young animals. However, prior treatment of the old animals by means of daily SFN gavage before and during sensitization prevented the response decrease and could restore the CHS response to the levels seen in young animals (FIG. 1, A). These response differences were maintained after 48 hours and were also reflected by histologic changes in the ear, which showed that the decrease in lymphocyte infiltration and intercellular edema in old animals could be reversed by means of SFN administration (FIG. 1, B). SFN had no effect on nonsensitized (control) animals.

IFN-γ and IL-4 message levels were measured in the ear tissues that were taken 48 hours after challenge to determine whether the induction of the CHS response is accompanied by polarized T cell differentiation. Quantitative RTPCR showed that DNFB challenge induced the expression of the T_{H}1 cytokine IFN-γ, which was significantly suppressed in old compared with young animals. SFN treatment significantly increased IFN-γ expression (FIG. 1, C). In contrast, the message level of a representative T_{H}2 cytokine, IL-4, was not significantly affected by aging or SFN administration (FIG. 1, C). In addition to the cytokine changes, message levels for T-bet, a T_{H}1-specific transcription factor, were significantly decreased in old versus young sensitized animals on DNFB challenge. SFN administration also prevented this decrease to a significant degree (FIG. 1, C). Neither aging nor SFN treatment had an effect on the expression of GATA-3, a T_{H}2-specific transcription factor.

The mRNA levels of p2Es (NQO1, glutathione S-transferase, γ-GCLS, and GPx) were determined by means of quantitative PCR to show that SFN affects p2E expression in vivo (FIG. 1, D). Compared with the expression levels in the livers of control animals, message levels for 3 of the 4 genes were increased by SFN administration (FIG. 1, D).

### Example 2

### Nrf2 Deficiency Accentuates the CHS Response Decrease in Old Mice

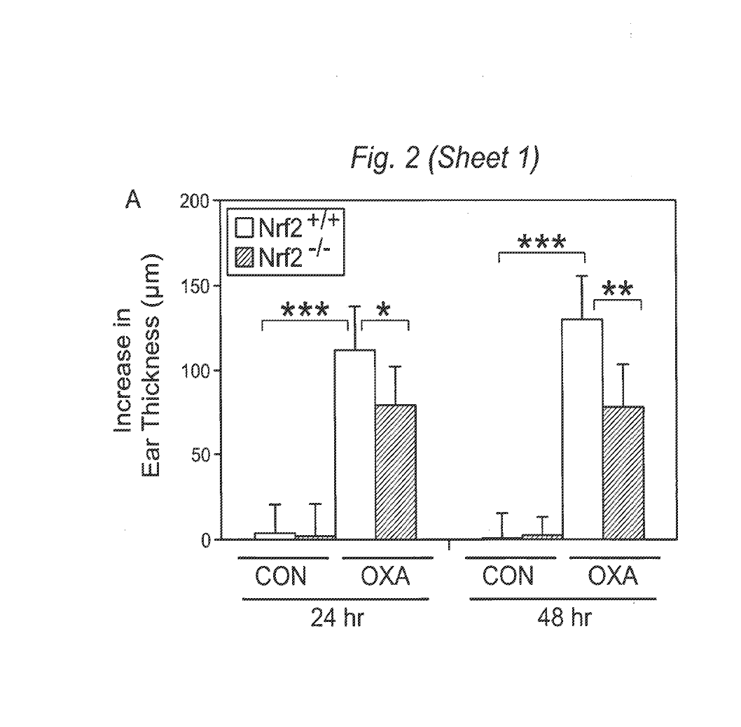

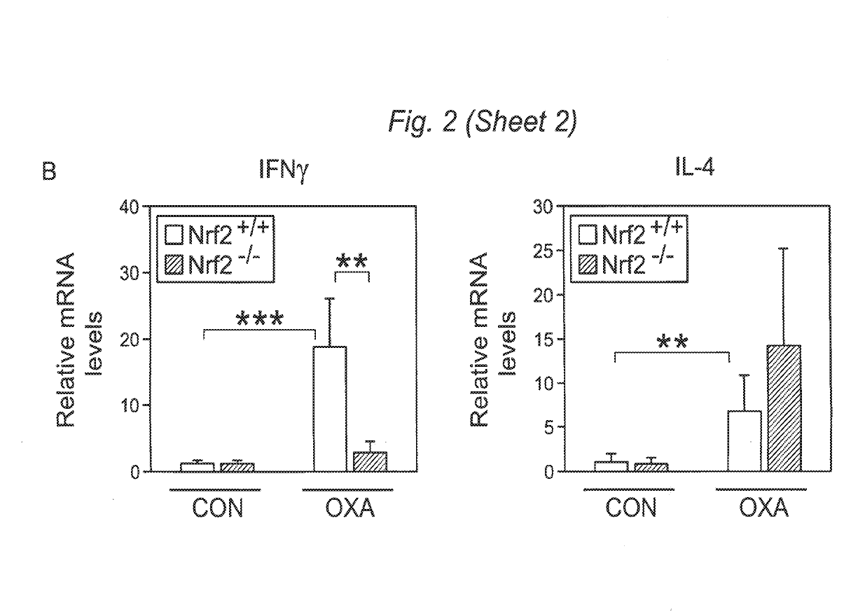

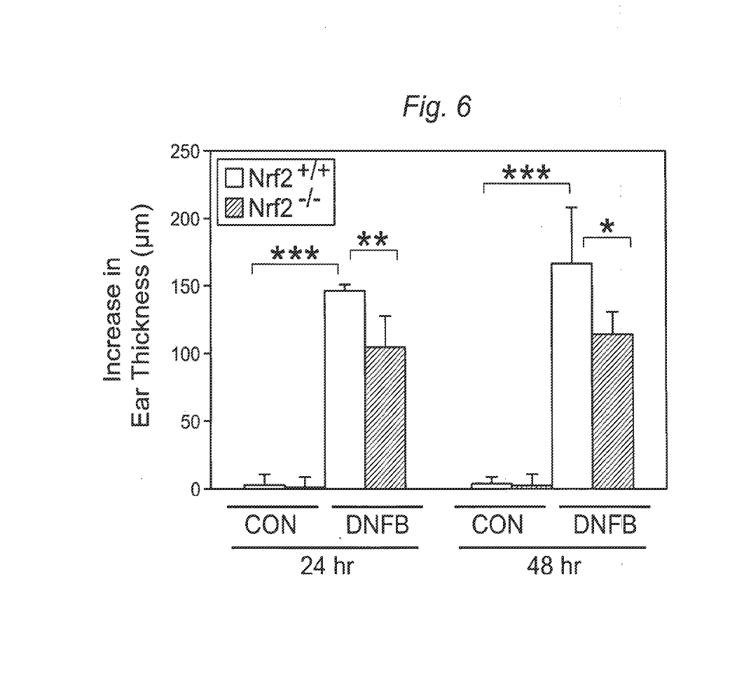

Nrf2 deficiency affects the immune function of old mice. To determine whether this includes an effect on T_{H}1 immunity and CHS, we compared the ear-swelling response of 22-month-old nrf2^{-/-} mice with littermate control animals (nrf2^{+/+} mice) during OXA sensitization and challenge. Nrf2-deficient mice showed a significant decrease in their ear-swelling response compared with that seen in wild-type control animals (FIG. 2, A). The same effect was observed when mice were sensitized and challenged with DNFB, indicating that the effect is not just limited to a single contact antigen (FIG. 6). This response reduction was accompanied by decreased IFN-γ mRNA expression, whereas IL-4 levels remain unaffected (FIG. 2, B). Interestingly, when this experiment was repeated in younger (6-month-old) animals, there was no response reduction in Nrf2-deficient mice. These results suggest that cumulative oxidative stress during aging accentuates the effect of Nrf2 deficiency in the immune system. These data suggest that through its ability to maintain redox equilibrium in the immune system, Nrf2 plays an important role in regulating T_{H}1 immunity, particularly under age-related oxidative stress conditions.

### Example 3

### DCs from Old Mice Contain Lower Levels of Phase II Enzymes (p2Es) and a Decreased Thiol Content

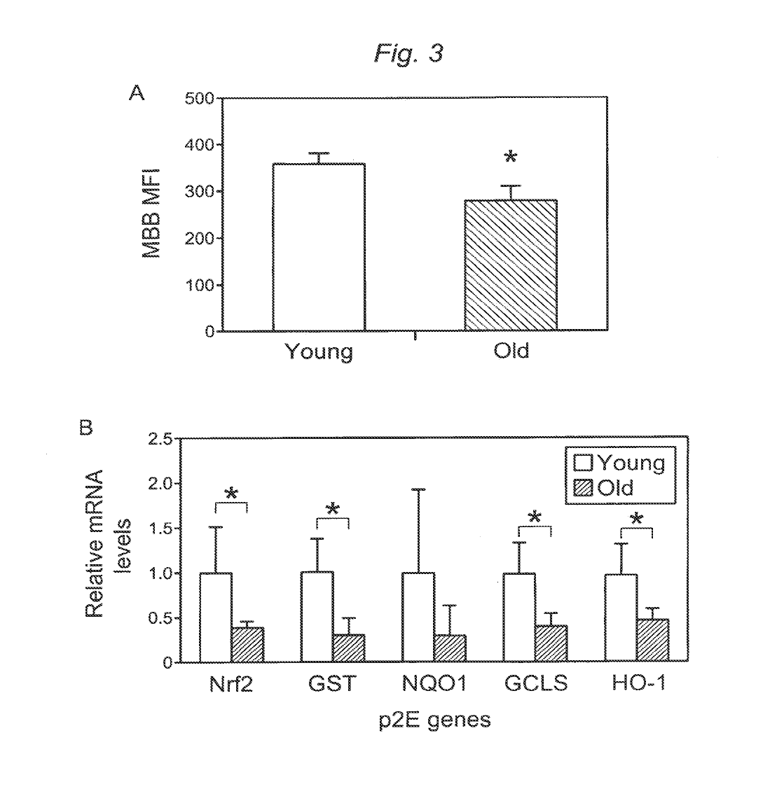

The CHS response involves several cell types in the skin, including helper T cells, cytotoxic T lymphocytes, and Langerhans cells (LCs). Although T cell function is clearly affected by the oxidative stress events during aging, increased ROS production also targets DCs. Due to the difficulty in obtaining a sufficient number of LCs to study the effect of changes in redox status, we compared thiol levels from CD11c⁺ cells that were purified from the spleens of young and old mice. This showed a significant decrease in MBB mean fluorescence intensity in the CD11c⁺ populations from old animals (FIG. 3. A). This decrease of 24% is highly significant from a homeostatic perspective because a small decrease in GSH content leads to a big decrease in the GSH/GSSG ratio. Similar observations were made when CD11c⁺ bone marrow derived DCs (BM-DCs) were compared in young and old mice. RNA was also isolated from purified CD11c⁺ cells to perform quantitative PCR analysis to assess p2E message expression. Decreased mRNA expression of p2Es and in nrf2 message were observed in cells from old animals (FIG. 3, B). These data indicate that aging leads to altered redox equilibrium in DCs and effects in APC function.

### Example 4

### DC Redox Disequilibrium Interferes in the CHS Response that can be Elicited by Adoptive Transfer of Myeloid DCs, Whereas the Restoration of DC Thiol Levels can Reverse this Effect

An adoptive transfer protocol previously demonstrated that antigen-pulsed BM-DCs from a donor elicit a CHS response in recipient animals (Kim et al., J Allergy Clin Immunol, 119:1225-33 (2007)). Moreover, we have demonstrated that GSH depletion of these DCs at the time of antigen processing leads to a reduced ear-swelling response in vivo.

One explanation for the impaired response is that oxidative stress decreases IL-12 and subsequent IFN-γ production in T cells. Through GSH synthesis and p2E expression, Nrf2 could modify the signaling pathways that are required for DC maturation, cytokine production, and costimulatory receptor expression. Another explanation is that DCs play an important role in neutralizing extracellular oxidative stress through the expression of surface thiol groups. Not only does this allow the DCs to survive in an oxidative stress environment, but it also contributes to the maintenance of thiol levels and viability in bystander T lymphocytes.

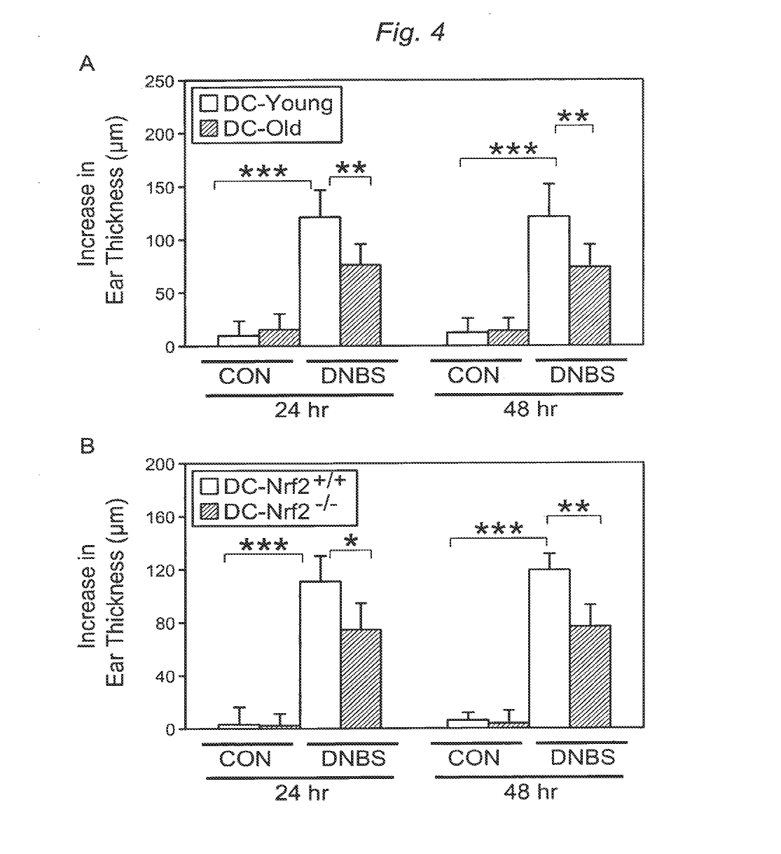

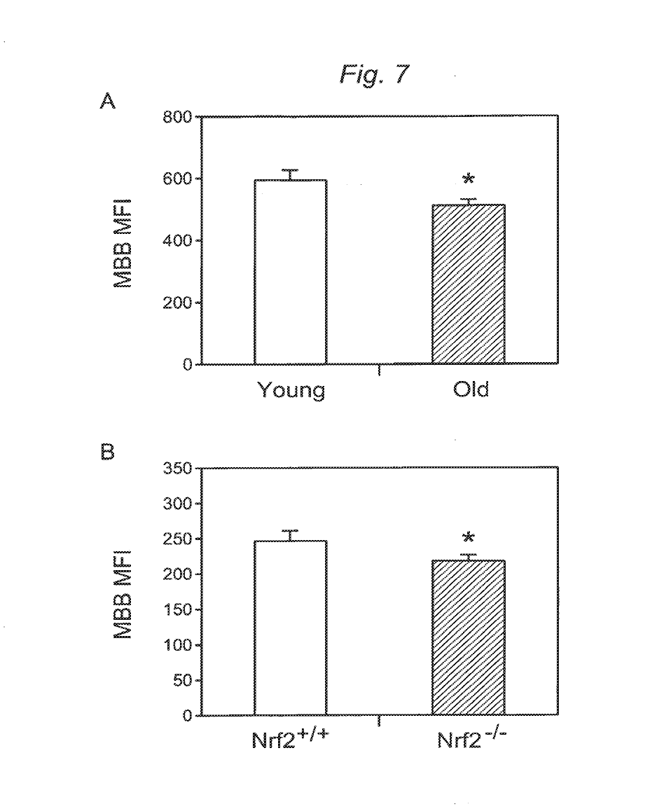

To investigate whether the age-related DC redox disequilibrium affects the adoptive CHS, BM-DCs from young and old mice were used for ex vivo pulsing with the water-soluble DNFB analogue DNBS. These cells were then subcutaneously injected into recipient young naive mice. Five days later, a CHS response was elicited by means of DNFB application to the ears of the recipient. A significant decrease in the ear-swelling response was observed when DCs from old animals were used compared to the younger counterparts (FIG. 4, A). Reduced inflammatory infiltrates in the ear tissue of old animals were also observed. No response was obtained in animals receiving naive DCs (control animals; FIG. 4, A). The data suggest that altered redox equilibrium is responsible for the decrease in DC function. We also performed MBB staining to look at BM-DC thiol levels. The small but significant decrease (14%, P<0.05) of total thiol levels in DCs from old animals could be responsible for a significant change in the GSH/GSSG ratio (FIG. 7, A).

The same experiment was performed with DCs from old nrf2^{+/+} and nrf2^{-/-} mice. FIG. 4, C shows that there is a significant decrease in the car-swelling response in mice receiving DNBS-pulsed DCs from nrf2^{-/-} compared with nrf2^{+/+} mice. This was accompanied by a significant reduction (13%, P<0.05) in the thiol content of BM-DCs from nrf2^{-/-} compared with nrf2^{+/+} mice (FIG. 7, B).

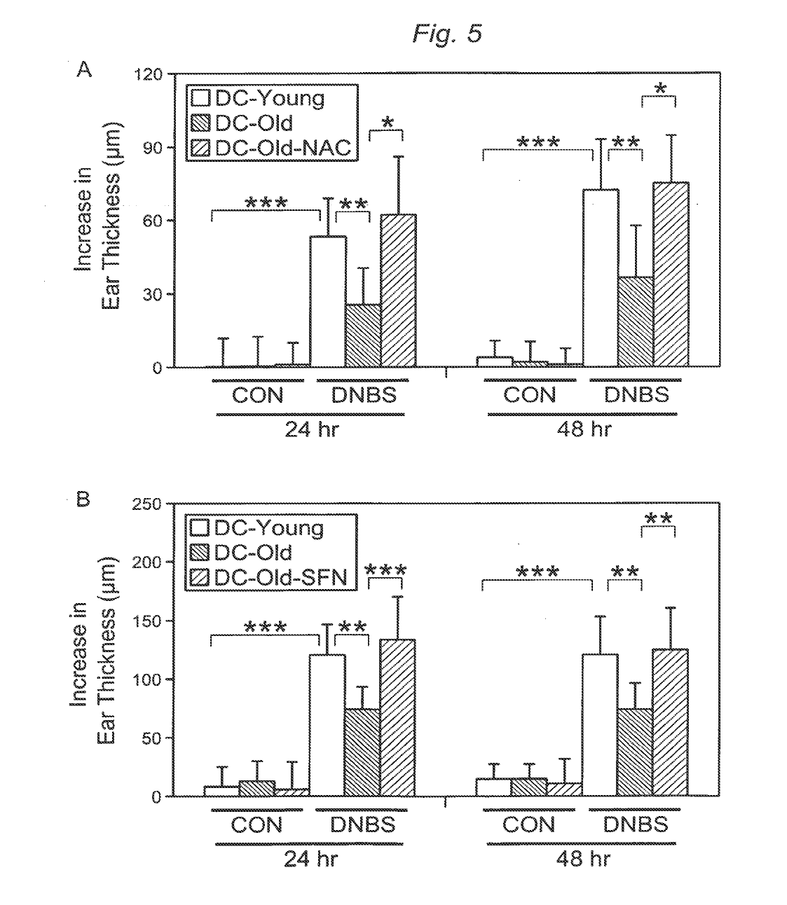

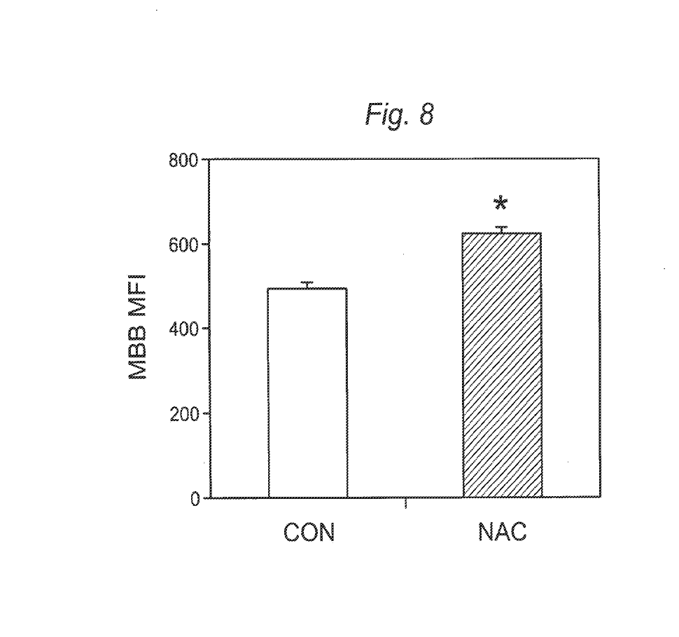

To confirm that the age-related changes in DC redox equilibrium is important for the maintenance of T_{H}1 immunity, we used the adoptive transfer approach to determine whether ex vivo thiol repletion could restore the CHS response. First, we confirmed that the ear-swelling response of recipient mice injected with DNBS-pulsed DCs from old animals was significantly decreased compared with that of mice injected with DCs from young animals. Second, we showed that ex vivo treatment with NAC restored the CHS response in the recipients (FIG. 5, A). MBB staining showed a 25% increase (P<0.05) in cellular thiol content with NAC exposure (FIG. 8). These data demonstrate that the age-related perturbation of DC redox equilibrium, thiol depletion, and nrf2 deficiency affect APC activity in vivo.

Assuming that most of the decrease in MBB fluorescence actually represents GSH conversion to GSSG, this would mean that converting 10% of GSH will result in a GSH/GSSG ratio of 9:1 to 2:1, whereas the corresponding ratio for a rate of 25% conversion will amount to 3:1. The ratio is generally 40:1 to 50:1 under normal redox conditions. Thus a small change in GSH content can have a big effect on the ratio of the GSH/GSSG redox couple that initiates cellular responses. Moreover, this decrease could lead to even more significant consequences during aging.

### Example 5

### SFN Treatment in DCs Reverses the Age-Related Decrease of CHS Response on Adoptive Transfer

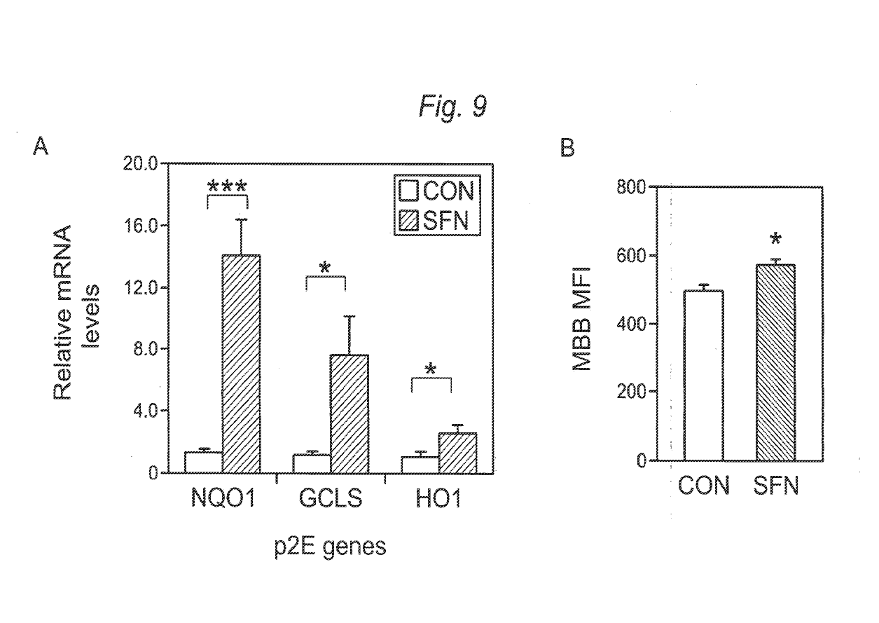

BM-DCs from old mice were treated with SFN before DNBS pulsing and injection into young recipient mice to determine whether SFN could exert similar effects in the DC adoptive transfer model as during oral administration (FIG. 1). Again, the CHS response to antigen-pulsed) DCs from old animals was reduced compared with the response to cells from young animals. Second, the data demonstrate that ex vivo SFN exposure could restore the CHS response elicited by DCs from old animals (FIG. 5, B). The results clearly indicate that activation of the Nrf2 pathway in DCs can reverse the age-related decrease in T_{H}1 immunity. As confirmation of upregulation of antioxidant enzyme expression in SFN-treated DCs from old animals, real-time PCR showed significant upregulation of the mRNA levels for NQO1 (P<0.001), γ-GCLS (P<0.05), and heme oxygenase 1 (P<0.05). This was accompanied by a 15% increase (P<0.05) in cellular thiol levels (FIG. 9).

There have been conflicting reports about the effect of thiol antioxidants on the CBS response (Särnstrand et al., J Pharmacol Exp Ther, 288:1174-84 (1999); Bruchhausen et al., J Invest Dermatol, 121:1039-44 (2003); Becker et al., J Invest Dermatol, 120:233-8 (2003); Su et al. J Immunol, 167:5084-91 (2001); Senaldi et al., J Invest Dermatol, 102:934-7 (1994)). There could be a number of reasons for these different outcomes, including differences in the experimental protocols, mouse strains, and contact-sensitizing chemicals used and different routes and times of antigen administration.

Our finding is supported by the study by Särnstrand et al., who showed a dose-dependent increase of ear thickness by NAC treatment in OXA-treated BALB/c mice. They also showed that DiNAC, an oxidized disulfide form of NAC, could enhance the CHS response. However, the CHS response induced by NAC is inhibited by the contemporaneous administration of DiNAC and vice versa. One can explain this interference by the thiol groups competing for covalent binding. Covalent binding to endogenous proteins constitutes one of the mechanisms by which contact sensitizers induce immune activation.

NAC has been shown to block the binding of the contact sensitizer TNCB to cellular proteins and prevented tyrosine phosphorylation (Bruchhausen et al.). This does not contradict our study because tyrosine phosphorylation could be indicative of oxidative stress that is reversed by an antioxidant. Our study differs from the Bruchhausen study in the time point at which NAC was administered. NAC administration at the time of TNCB sensitization creates the possibility of NAC binding to the chemical, thereby interfering in binding to cellular proteins. In our study DCs were pretreated with NAC, which was washed away before adding the contact sensitizer (FIG. 5, A).

Different types of thiol-modulating compounds can affect the CHS response differently in different mouse strains. C57BL/6 mice are considered to have a T_{H}1 phenotype, and BALB/c mice are known to be more T_{H}2 prone. Särnstrand et al. showed that DiNAC augmented the OXA response while decreasing the CHS response in BALB/c mice. Senaldi et al. (J Invest Dermatol, 102:934-7 (1994)) demonstrated that orally administered NAC (1.6 g/kg) reduced the TNCB response in BALB/c mice. Venkatratnan et al. (Arch Dermatol Res, 296:97-104 (2004)) reported that thiazolidinedione derivatives of the α-lipoic acid inhibited allergic contact dermatitis in OXA-treated NMRI mice. In contrast, we showed that NAC increases the CHS response to OXA and DNFB in C57/BL6 mice (FIG. 5, A).

In summary, intervention through the Nrf2 pathway provides a rational approach to improve cellular immune function during aging. In addition to the beneficial effects on specific immunity, it is possible that many of the chronic inflammatory changes that develop in the elderly might originate in the innate immune system. An age-related decrease in Nrf2 activity can lead to oxidative stress-mediated proinflammatory responses in cells from the innate immune system. Thus, Nrf2 agonists can also be used intervene in this aspect of aging.

1.-6. (canceled)

7. A method of improving the efficacy of a vaccine in an aging individual in need thereof, comprising: administering to the individual a vaccine and a nutraceutical composition comprising an effective amount of a Nrf2 pathway agonist (NPA), thereby improving the efficacy of the vaccine in the individual.

8. (canceled)

9. The method of claim 7, wherein the NPA is selected from the group consisting of sulforaphane, glucoraphanin, or α-linoic acid.

10. The method according to claim 7, wherein the vaccine is an influenza vaccine, a rabies, a hepatitis A, a hepatitis B, a hepatitis C, a human papilloma virus, a polio, a mumps, a measles, a rubella, a diphtheria, a pertussis, a tetanus, a HiB, a chickenpox, a rotavirus, a meningococcal disease, or a pneumonia vaccine.

11.-17. (canceled)

18. The method of claim 7, wherein the NPA is an NPA-containing food or food extract.

19. The method of claim 7, wherein the NPA is extracted from a plant or another cruciferous vegetable.

20. The method of claim 7, wherein NPA is sulforaphane (SFN).

21. The method of claim 7, wherein the nutraceutical composition comprises two or more NPAs.

22. The method of claim 21, wherein the two or more NPA are from two or more different sources.

23. The method of claim 7, comprising administering two or more vaccines.

24. The method of claim 7, wherein the nutraceutical composition further comprises a compound to restore redox equilibrium.

25. The method of claim 24, wherein the compound to restore redox equilibrium is N-acetyl cysteine (NAC).

26. The method of claim 7, wherein the aging individual is at least 30 years old.

27. The method of claim 7, wherein the aging individual is at least 40 years old.

28. The method of claim 7, wherein the aging individual is at least 50 years old.

29. The method of claim 7, wherein the aging individual is at least 60 years old.

30. The method of claim 7, wherein the improved efficacy of the vaccine is assessed by an increase in T_{H}1 function.

31. The method of claim 7, wherein the improved efficacy of the vaccine is assessed by an increase in phase II enzyme expression in immune cells.

32. The method of claim 7, wherein the improved efficacy of the vaccine is assessed by improved recruitment of antigen presenting cells to sites of injury or inflammation.

33. The method of claim 7, wherein the antigen presenting cells are dendritic cells.

34. The method of claim 7, wherein the improved efficacy of the vaccine is assessed by improved antigen presenting cell activity.

35. The method of claim 7, wherein the antigen presenting cell activity is dendritic cell activity.

36. The method of claim 7, wherein the improved efficacy of the vaccine is assessed by improved antigen response.

37. The method of claim 7, wherein the improved efficacy of the vaccine is assessed by improved innate immunity.

38. The method of claim 7, wherein the NPA is administered in the same composition as the vaccine.

39. The method of claim 7, wherein the NPA is administered in a separate composition from the vaccine.

40. The method of claim 7, wherein the NPA is administered orally, by inhalation, by transdermal means, or by injection.

41. The method of claim 7, wherein the vaccine is administered orally, by inhalation, by transdermal means, or by injection.

42. A method of improving the efficacy of a vaccine in an aging individual in need thereof, comprising: co-administering to the individual a nutraceutical composition comprising an effective amount of a Nrf2 pathway agonist (NPA) and a vaccine, thereby improving the efficacy of the vaccine in the individual, wherein the NPA is SFN.

43. A method according to claim 42, wherein the SFN is administered orally.

44. The method according to claim 42, wherein the vaccine is an influenza vaccine, a shingles vaccine, a chickenpox vaccine, or a pneumonia vaccine.
